(19) United States
(12) Patent Application Publication
(10) Pub. No.: US 2005/0222959 A1
(43) Pub. Date: Oct. 6, 2005

(54) Method for marketing rights to intellectual assets

(76) Inventor: Barry Brager, Smyrna, GA (US)

Correspondence Address: Eduardo M. Carreras, Suite 102, 768 Marietta Street NW, Atlanta, GA, 30318, US

(21) Appl. No.: 11/094,960

(22) Filed: Mar. 31, 2005

Related U.S. Application Data
(60) Provisional application No. 60/558,604, filed on Apr. 1, 2004.

(51) Int. Cl. H04K 1/00; H04L 9/00; G06F 17/60; A61K 31/53; A01N 43/66

(52) U.S. Cl. 705/54

(57) ABSTRACT

Disclosed is a system and method for the marketing and bulk optioning, licensing and selling of intellectual assets via a virtual and/or physical event involving multiple participants. Events are organized to identify and optimize intellectual asset packages and present them in standardized forms in order to efficiently facilitate multiple licensing opportunities. Participants are provided with the opportunity to view presentations and then communicate with presenters in a private setting during the event to initiate or consummate deals. Compensation to an event managing party includes contingency fees based on the volume of transactions resulting from the event.

### RELATED CASES

This application claims the benefit of commonly owned Provisional Patent Application Ser. No. 60/558,604, filed Apr. 1, 2004. The disclosure of the foregoing Provisional Patent Application is incorporated in its entirety herein by reference. Priority benefit of aforementioned Provisional Patent Application is hereby claimed.

### TECHNICAL FIELD OF THE INVENTION

This invention relates generally to the methods for the transfer of rights to intellectual assets (“IA”) and in particular to methods for marketing the IA.

For the purpose of this invention IA include, among other things, information, including trade secrets, know-how and other valuable information; inventions, including processes, machines, manufactures, or compositions of matter; utility patents and patent applications; design patents and design patent applications; plant patents and plant patent applications; trademarks and service marks; original works of authorship fixed in a tangible medium of expression; including literary works, musical works, including any accompanying words; dramatic works, including any accompanying music; pantomimes and choreographic works; pictorial, graphic, and sculptural works; motion pictures and other audiovisual works; sound recordings; and architectural works; copyrights, mask works, utility models and similar rights that are granted by countries to protect the original works of individuals.

Intangible assets, including IA have come to represent a significant portion of a company's value. As a result, companies, inventors and asset owners have invested significant amounts of time, money and resources to develop and prosecute their IA. In turn, numerous organizations have developed licensing teams and/or divisions in order to exploit and derive revenue to offset the costs of building their IA portfolios. These teams and divisions, however, tend to focus on revenues that are perceived to be more certain to obtain, such as those from potential licensees already manufacturing or selling products encompassing protected IA (“infringers”). “Assertive licensing” is the specific term used to force infringers to pay or discontinue sale of products believed to infringe. However, assertive licensing is expensive, as it typically requires the gathering of sparse competitive intelligence, performing reverse engineering, and in some cases, time-consuming litigation.

Published U.S. patent application Ser. No. US20020152146A1 (Reader, Oct. 17, 2002) discloses a method for identification of candidate and qualified patent licensing targets through the expedient of assessing technological overlap between patent assets of the licensing entity and third party entities. By using third party patenting activity to drawn inferences about third party business activity, the invention allows a threshold identification of third parties as candidate licensing targets to be made and a further identification of candidate licensing targets as qualified licensing targets to be made without requiring any direct knowledge concerning third party business activity. Moreover, the present invention may be applied in a networked computing environment to allow such identifications to be made with relatively small cost and time commitment.

U.S. patent application Ser. No. US20010044781A1 for a “Computer implemented and/or assisted method and system for facilitating the licensing of media content” (Shutes, Nov. 11, 2001) discloses an Apparatus and a Process to expedite the licensing and management of content for communications projects, including books, magazines, newsletters, posters, advertisements, videos, films, television, CD ROMs, digital media, multimedia, and other content. The Apparatus allows the User to expedite several of the administrative tasks necessary to research, obtain, track and license content. In addition, it allows the User to quickly determine the technical and contractual viability of potential designs or drafts of the project. The Process allows the User to streamline negotiation, implementation, and payment of licensing contracts by efficiently gathering, organizing, and utilizing project information, technical specifications, and/or licensing terms. It also assists the User in tracking budgets and managing resources within the production of the project. The electronic portion of the Apparatus can be accessed using a personal computer, an internal network and a server, an external network and a server, dial-up system, or other electronic communications method. The electronic portion of the Apparatus will also be able to communicate and/or integrate with third party software used to design and edit communications projects.

IA, however, appeal to a broader group of users than just those who may be caught infringing. Recent economic shifts have permitted some companies to gain funding for endeavors that may benefit from the voluntary (“opportunistic”) licensing of the IA of others. In addition, opportunistic licensing is an ideal foreign market entry alternative to direct exports or foreign direct investment.

U.S. patent application Ser. No. US20020004775A1 for an Online patent and license exchange (Kossovsky, Jan. 10, 2002) discloses an online patent and license exchange is provided which enables potential licensors and licensees of patents and other intellectual property rights to efficiently and reliably transact IP license or assignment agreements. The online patent and license exchange includes a method of providing a valuation of an IP asset using data from companies in a similar technology classification as the IP asset, a method of generating a suggested IP asset asking price, an index of IP market value according to technology classification, a method for quantifying the financial risk of an asset (β), and a quantitative database of technology attributes of IP assets.

Regardless of whether a licensing scenario is assertive or opportunistic, the IA of an organization are typically not bundled or packaged for sale in any systematic or optimized manner. Typical licensing endeavors are related to single patents, trademarks or copyrighted materials. Or, they are organized along product lines. There have been occasional patent or IA “pools” assembled from a plurality of owners, in order to optimize licensing timelines and revenues. However, these pools are not common and have historically been organized by entrepreneurs or industry consortia, as opposed to the asset owners.

Perhaps most important, the licensing of IA has been only a marginally successful process for the asset owners. Due to the fact that the process relies heavily on personal networking, timelines required to close deals are inordinately long. In addition, companies rarely look beyond their own industry to market IA. As a result, the current practice yields only a few deals per company annually, with high transaction costs.

The licensing and rights transfer methods in use today substantially ignore the role of marketing. In general, marketing activities are all those associated with identifying the particular wants and needs of a target market of customers, and then going about satisfying those customers better than the competitors. This involves doing market research on customers, analyzing their needs, and then making strategic decisions about product design, pricing, promotion and distribution. This approach can be applied to IA.

Another drawback of existing approaches to licensing is that they do not provide potential transferees the opportunity to access the information and knowledge necessary to commercialize the IA and which are not necessarily disclosed in say a patent. Another disadvantage is that the existing methods are primarily conducted by the asset owners, and do not have the advantage of a disinterested third party view of the assets and their value.

Organizations, therefore, have not fully captured all of the opportunities to maximize revenues associated with their IA. In addition, the organization handles these tasks separately from the inventors, providing yet another area of inefficiency.

Therefore, there is a need for a method of transferring right to IA that applies marketing disciplines to package the IA in a way that satisfy the potential transferee's wants and needs.

There is also a need for a system and method which provides an organization or grouping of organizations with a method of opportunistically optioning, licensing or selling IA in bulk, via a singular event, to permit the organization to more efficiently exploit and extract revenue from IA, with lower transaction costs. Such a system should be able to accommodate a variety of asset types and configurations, in a variety of developmental stages, and which can be integrated into a well-managed licensing process associated with an organization's IA management practices. The system should also include a means for organizing and optimizing assets from a market perspective, and to attract licensees from both inside and outside the assignee's industry and/or geography. Further, the system should facilitate consistent presentations, communication and deal making, as events are inherently marketing activities and attendees may certainly be viewed as prospective customers.

There is also a need for a transaction cost management method when creating such events, since IA options, licenses and sales are inherently more risky than other transactions. It is therefore favorable to design compensation into the system that provides incentive for successful outcomes from such an event, and which can reduce costs in event of minimal interest.

### SUMMARY OF THE INVENTION

According to one example embodiment of the invention, there is provided a method of marketing rights to IA that includes the step of organizing an event where the owners of the IA can present the assets to be transferred and ancillary information to at least one potential transferee in a standardized format.

According to another feature of the invention, the IA rights to be transferred are selected and aggregated according to predetermined criteria.

In another embodiment, a Facilitator is selected to organize and manage the event where the IA rights to be transferred are presented.

In still another embodiment, the Facilitator is compensated in one or more of the following ways: requiring a participant in the event to pay an attendance fee; requiring a sponsor of the event to pay a sponsorship fee; requiring the IA Owner to pay a percentage of the proceeds of a transaction as a fee.

In yet another embodiment, the method further comprises the step of selling shares to the Facilitator's compensation.

### BRIEF DESCRIPTION OF THE DRAWING

FIG. 1 illustrates the basic steps of an assertive licensing method commonly used by IA owners and which is part of the prior art.

FIG. 2 illustrates the basic steps of the method of the present invention.

FIG. 3 illustrates the additional steps and activities involved in the selection of IA rights for transfer according to the present invention.

FIG. 4 illustrates the additional steps and activities involved in the preparation of presentations.

FIG. 5 illustrates the additional steps and activities involved the organization of an event in accordance with the invention.

FIG. 6 illustrates an alternative method according to the invention and the collection of fees.

FIG. 7 illustrates yet another embodiment of the method of the invention.

FIG. 8 illustrates a method where interests in the income stream from the transactions are sold to third parties.

### DETAILED DESCRIPTION OF THE INVENTION

In the following detailed description of the preferred embodiments, reference is made to the accompanying drawings that form a part hereof, and in which are shown by way of illustration specific embodiments in which the invention may be practiced. It is understood that other embodiments may be utilized and structural changes may be made without departing from the scope of the present invention.

Introduction

The present invention provides an IA owner with the ability to market rights to the IA to potential transferees in an event which allows the delivery of the information necessary for a potential transferee to make a decision about acquiring the rights.

A useful property of the marketing method is that it facilitates the exchange of information between the IA Owner and potential transferee so that the interests of both parties can be harmonized.

### EXAMPLE EMBODIMENT OF THE INVENTION

For simplicity and illustrative purposes, the principles of the invention are described by referring mainly to an exemplary embodiment thereof. However, one of ordinary skill in the art would readily recognize that the same principles are equally applicable to, and can be implemented in, a system capable of developing a wide range products, services and content associated with business opportunities, and that any such variations are within the scope of the invention. While in the following description numerous specific details are set forth in order to provide a thorough understanding of an embodiment of the invention, in other instances, well known methods and structures have not been described in detail so as not to obscure the invention.

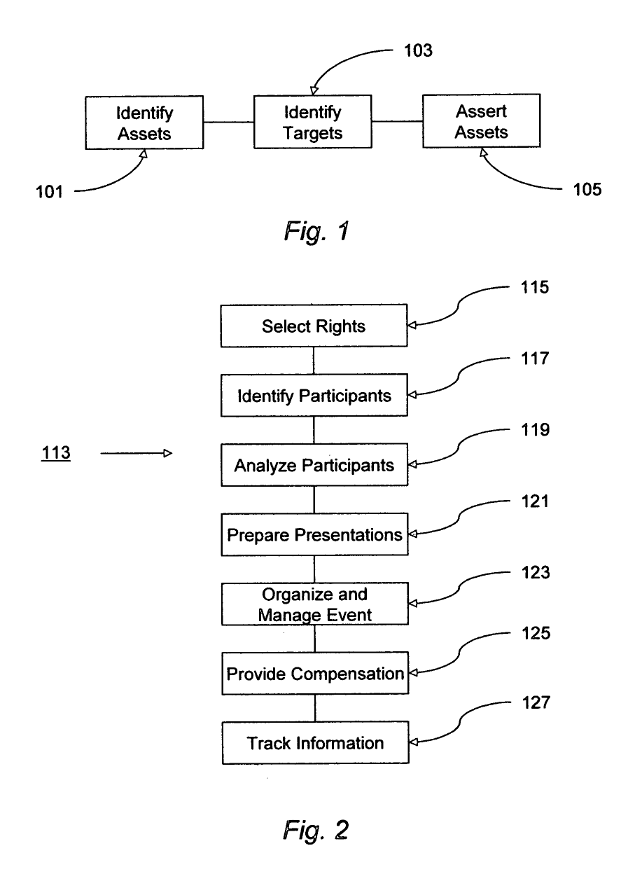

FIG. 2 is an exemplary diagram of a method 113 for marketing rights to IA. At a high level, the method comprises the steps of selecting the rights to be transferred 115, identifying participants (i.e. potential transferees) 117 (Identify Participants), analyzing the participants 119 (Analyze Participants), preparing presentations 121 (Prepare Presentations), organizing and managing an event 123 (Organize and Manage Event) where details of the rights to the IA property to be transferred are presented to the participants, providing compensation 125 (Providing Compensation) and tracking information about potential transactions 127 (Track Information). Although the in the flow chart in FIG. 2 the steps appear sequential, it is likely that some of the steps may be performed in parallel. For example, step 123 (organize and manage event) may be started even before step 115 (select rights) is completed. The event referred to in step 123 (Organize and Manage Event) is designed for the express purpose of transferring rights of IA owned by at least 1 participant in the event to at least 1 other participant of the event and for generating revenues from option fees, licensing fees or sales fees.

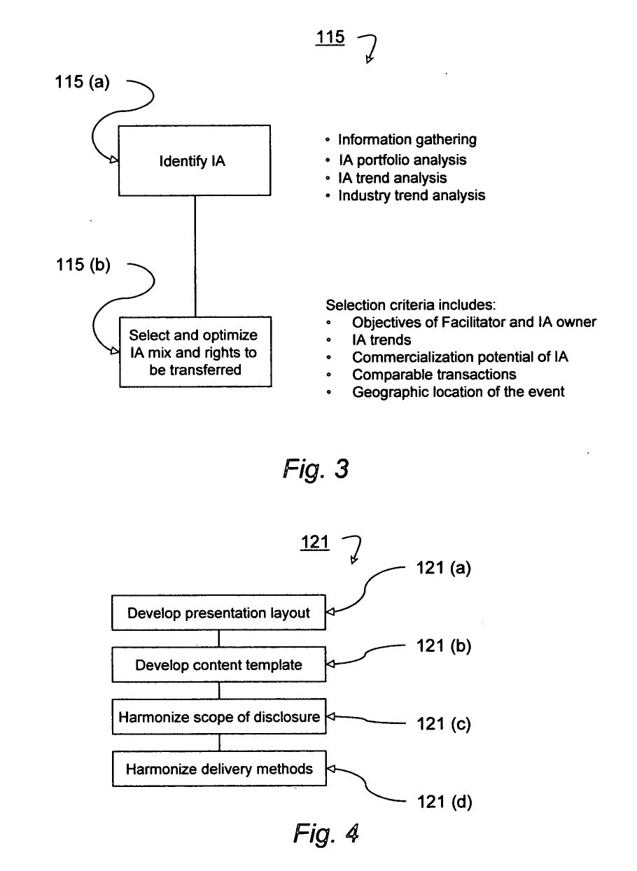

Step 115 (Select Rights) may require certain activities to be performed by a party that desires to transfer the rights to the IA (“Transferor”). The Transferor may be the owner of the IA or a licensee of those assets having the right to transfer some of its rights to another party. Step 115 also may require certain activities to be performed by a party (“Event Facilitator”) that will plan and manage the event in step 123 (Organize and Manage Event). Illustrated in FIG. 3 are the basic steps in step 115 (Select Rights), which include step 115(a) (Identify IA) and step 115(b) (Select and Optimize IA Mix and Rights to be Transferred). Step 115(a) (Identify IA) is an information gathering and analysis step. As illustrated in FIG. 3 among the activities to be performed may be: information gathering, including interviewing event participants, interviewing legal counsel of at least 1 event participant, interviewing external legal counsel unrelated to any event participant, interviewing employees of at least 1 event participant, interviewing competitors of at least 1 event participant, interviewing licensees of at least 1 event participant, interviewing prospective licensees of at least 1 event participant and/or interviewing customers of at least 1 event participant. Another set of activities that may be performed in step 115(a) (Identify IA) is to conduct IA portfolio analysis. An IA portfolio analysis may include: conducting quantitative analysis of a portfolio of at least 1 IA, conducting qualitative analysis of a portfolio of at least 1 IA, conducting legal analysis of a portfolio of at least 1 IA, and/or conducting financial analysis of a portfolio of at least 1 IA. Quantitative analysis may include for example in the case of patents, how many patents are held by the IA Owner for each relevant class and how many patents are in the class, how many patents are owned by competitors and similar information of a quantitative nature. Qualitative analysis of a portfolio may include, again in the case of patents as an example, scope of the claims, when the patents expire, scope of coverage of ancillary technology, identification of non-infringing substitutes, etc. Legal analysis may include again in the case of patents, an analysis of issues related to the enforceability of the claims, defects in the chain of title, validity analysis and the like. Finally, conducting a financial analysis may, in the case of a patent include valuation, sizing the market opportunity for goods and services covered by the claims of the patent, identification and analysis of profit margins for covered goods, determining the price elasticity of the covered goods and services, etc. Another activity that may be conducted in step 115 (a) is to perform an IA trend analysis. An IA trend analysis may entail reviewing the IA registration trends of one or more relevant countries, for example the filing and issuance rate of patents for the relevant classes for a period of time. The activity may also include the review of filing data for one or more inventors in related patents or the trademark registration trends of a particular trademark office or a particular trademark owner. Although some of the specific examples of the activities referred to above are related to patents, it is understood that any IA for example, copyrights, trademarks, and trade secrets may be subject to similar analysis.

The output of step 115(a) (Identify IA) will be information that will be used in step 115(b) (Select and Optimize IA Mix and Rights to be Transferred). Included in the information will be selection criteria including the objectives of the Facilitator. These objectives will take into account the financial strategies of the IA Owner and the Facilitator or any potential for a merger or acquisition of a potential transferee or other relevant party. Consideration may be given to the IA trends identified in step 115(a) (Identify IA) as well as the commercialization potential of the IA, any information about comparable IA transfers and geographic considerations. In developing the objectives the Facilitator and the IA Owner may take into account competitive intelligence obtained on any potential transferee, or other participant in the event. The objective of step 115(b) is to create the most marketable, licensable or sellable packages of assets—each package comprised of at least 1 IA for option, license or sale.

Step 117 (Identify Participants) is an important step in the method of the present invention. In step 117 (Identify Participants) the objective is to create a list of all parties that may be potentially interested in obtaining rights to the IA. That list may be compiled by: seeking recommendations from the originators of the IA or the IA owner; seeking recommendations from existing IA licensees; seeking recommendations from employee of the IA Owner or its licensees; seeking recommendations from prospective event participants; seeking recommendations from competitors of the IA Owner. Also included in the list would be competitors of the IA Owner, partners of the IA Owner; and owners of similar or complementary IA (e.g. patent holder in a similar or complementary primary patent classification as the IA). In cases where the IA includes patents potential participants may include patent holders referenced in a reverse citation of the IA, or patent holders referenced in a forward citation of the IA. Potential participants may also include parties identified as belonging to the industry of IA Owners or other potential participants that have been identified. For example, potential participants may include at least 1 participant in at least 1 SIC code matching at least 1 other participant in the event; at least 1 participant in at least 1 industry type matching at least 1 other participant in the event. Other criteria for identifying potential participants include geographic area, financial rating, common legal representatives, common designated agents; common employees; or participation in similar trade associations or advocacy groups. Other potential participants may be identified from groups having a similar research environment as at least 1 other participant in the event; similar academic environment as at least 1 other participant in the event; groups belonging to a similar governmental organization as at least 1 other participant in the event. Yet another way of identifying potential participants is through market research of least 1 assigned asset to be presented at the event. The output of step 117 (Identify Participants) is a comprehensive list of potential participants to the event.

Once the list of potential participants is created some analysis of the potential participants as in step 119 (Analyze Participants) is desired. The analysis step may include simple tasks such as compiling information about key personnel, addresses, e-mail addresses, industry data and the like. The output of this step would be a summary of information related to each potential participant with insights about their wants and needs with respect to the IA.

An important step in the process of the present invention is step 121 (Prepare Presentations) illustrated in FIG. 2 and illustrated in more detail in FIG. 4. The output of step 121 (Prepare Presentations) will be one or more presentations that address the needs and wants of the potential transferee while at the same time substantially meeting the objectives identified in step 115 (Select Rights). A presentation may be created for each package of IA identified in step 115 (Select Rights). As illustrated in FIG. 4, the step 121 (Prepare Presentation) may include step 121 (a) (Develop presentation layout); step 121(b) (Develop content template); step 121(c) (Harmonize scope of disclosure); and step 121(d) (Harmonize delivery methods). The objective of step 121(a) is to develop presentation(s) that are preferably consistent in formatting of design, layout, color and typography. The objective of step 121 (b) (Develop content template) is to ensure that the organization of the presentation will be preferably consistent in terms of the relevant information presented, including: description of presenters; description of innovators and/or inventors; description of the IA(s); the advantages to be derived from the rights offered under the IA. If the IA relates to innovations the presentation(s) the content template preferably will include: a description of the innovation(s) related to and/or contained in the package of IA(s); a timeline of the innovation(s); scientific data supporting the advantages of the innovation(s); summary of improvements over the prior art; an analysis of the risks associated with the innovation(s); remaining work to fully commercialize the innovation(s); Specific IA and/or property rights associated with the innovation(s) and the like. The presentation(s) may also include general terms related to optioning, licensing or selling the package and further information for consummating a deal with the owner of the package of the IA(s).

An important objective of step 121 (Prepare Presentations) is to ensure that the level of disclosure of confidential information is consistent and controlled. This is accomplished by step 121(c) (Harmonize scope of disclosure). In this step, the Facilitator and IA Owner will: monitor the volume of confidential information conveyed; monitor and enforce compliance with the security and/or disclosure guidelines of the IA Owner; and monitor and enforce compliance with applicable laws and regulations.

Another important objective of step 121 (Prepare Presentations) is to deliver the information relating to the IA in a consistent method and/or style. This is accomplished by step 121 (d) (Harmonize delivery methods). This is accomplished by coaching presenters in advance of their presentation; providing verbal and/or written feedback to presenters to improve their presentations prior to and/or during an event; and providing training documents with best practices and advice to presenters prior to an event. Additional ways that such consistency may be achieved may include: creating an online collaborative environment where presenters can communicate with each other; creating an online collaborative environment where presenters can communicate with at least 1 other event participant and providing incentives to presenters to be consistent though monetary and/or non-financial rewards.

The resulting presentation(s) may be posted in an electronic format with appropriate security to facilitate annotation, correction and comment by event participants. Such a posting could also be employed as a source of information to event participants in case of bidding. Other functions that such a posting may serve include forwarding the presentation(s) to approved recipients, disclosing purchase intent by event participants or facilitating the ranking of the presentation(s) or the IA assets by reviewers or event participants. Another function that electronic posting would serve is to promote direct electronic contact between the originator of the IA (e.g. inventor) and the event participant or alternately between the IA Owner and a potential transferee. Other functions may include electronic contact between reviewers and event participants and other reviewers and event participants. The posting of the presentation in electronic format may also facilitate legal review of the IA and presentation. Other functions of the posting of the presentation in electronic format may include: assessment of asset option, licensing or sale status by reviewers and/or event participants; assessment of asset option, licensing or sale value by reviewers and/or event participants; and delivery of an asset to reviewers and/or event participants. The presentation may be modified as a result of the communications facilitated by the electronic posting.

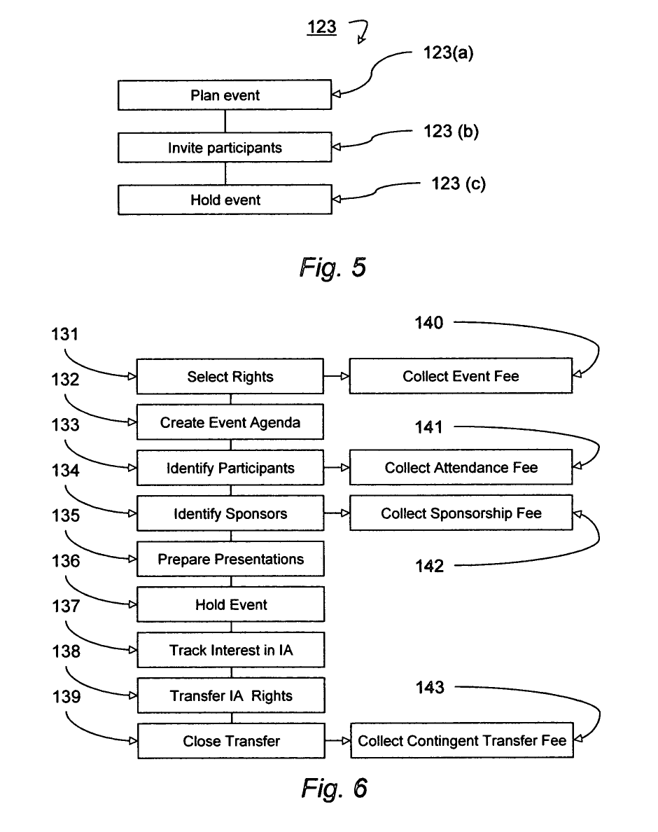

As illustrated in FIG. 5 step 123 (Organize and Manage Event) includes a planning step 123(a) (Plan Event) an invitation of participant step 123 (b) (Invite participants) and an execution step 123(c) (Hold Event). The event may be a conference, meeting or gathering of participants for the intent of evaluating IA offered for option, license or sale. The event may be a physical event or a virtual event or a combination of physical and virtual components. Participants in the event may include a Facilitator, who will manage the event, IA Owners, potential transferees, other interested parties which may include industry players, sponsors of the event, and competitors; and employees of the aforementioned parties. Step 123(a) (Plan Event) would include all activities relating to event management planning, such as selecting the date, venue, arranging for space and accommodations if a physical meeting or for a website or other technology if the event is a virtual meeting. Other activities in meeting planning may include arranging for audiovisual equipment, meals. Other activities may include budget development, promotion and publicity and the like.

Another step relating to step 123 (Organize and Manage Event) is the step of inviting potential participants 123 (b) (Invite participants). This may be accomplished through regular mail or electronic means.

Holding the event, step 123 (c) (Hold Event) entails a number of activities specific for the purpose of the event. These include: providing a virtual and/or physical forum for participants in order to deliver presentations developed in step 121. Part of the objective of step 123 (c) (Hold event) is to permit participants to indicate interest in IA during and/or after presentations. This can be accomplished by: electronic means, such as Email, telephone, remote control, instant message, Website visit, vote or video communication. Participants may also indicate interest by physical means, such as: requesting that the Facilitator set up a side meeting, a message on paper, ballots, bidding cards, list, raised hand and surveys. Another objective of step 123 (c) (Hold event) is to permit those participants expressing interest in IA to meet with presenters and/or other participants. This can be accomplished in a physical meeting, a virtual meeting or a combination of a physical and virtual meeting. These meetings would be facilitated and are intended to provide private interaction between parties and expedite the execution of non-disclosure agreements. Opportunities to describe in-depth IA-specific information for at least 1 IA would be provided during the event. The specific information may include inventor history, intellectual property prosecution history, legal history, research history, scientific data, commercialization timeline, commercialization requirements, commercialization potential, applicable industries, applicable inventions, future development plans, known risks, and associated know-how. These meetings may also provide an opportunity to present transaction specific terms such as terms license or sale, including options, timelines and pricing. The event may optionally provide means to facilitate the closing of a transaction by providing for example access to file histories, financial disclosures, and required paperwork. Additionally the event may provide the ability to meet with authoritative parties, intermediaries, brokers or negotiators.

If no deal can be documented at the event or during the event date(s), means may be provided to track interest in specific IA expressed at the event via physical and/or electronic means. Among the tracking data to be included in the means for tracking would be the name of interested party/parties, the assets/packages of interest, details of discussions, records of non-disclosure signings, asset uses indicated, timelines indicated, prices/values indicated, terms indicated, milestones indicated, commercialization objectives indicated and know-how required.

When a Facilitator provides event management services such services typically will be compensated. Step 125 (Provide Compensations) in FIG. 2 may include: a fee determined by projected and/or actual revenues generated from relationships created in the course of designing, planning and executing the event. Those revenues would result from a completed transaction involving the IA. Alternately compensation for services may include: an event management fee; a per-attendee fee; a per-sponsor fee; a per-license fee or a per-deal (other than license) fee. FIG. 6 illustrates the ways that the fees identified above may be collected. In FIG. 6 the method 113 comprises step 131 (Select Rights); step 132 (Create Event Agenda); step 133 (Identify Participants); step 134 (Identify Sponsors), step 135 (Prepare Presentations); step 136 (Hold Event), Step 137 (Track Interest in IA) where information about potential transferees id tracked; step 138 (Transfer IA Rights) where negotiations regarding a transaction for the transfer of IA rights is concluded; and step 139 (Close Transfer) where the transaction is closed and the transferee pays the IA Owner for the rights. An Event Fee 140 may be collected at the time the IA owner agrees to participate. An attendance fee 141 may be collected when participants are identified, invited and agree to participate. If the Event is sponsored by third parties a sponsorship fee 142 may be collected when the sponsors are identified. Finally a transfer fee 143 may be collected when a transaction closes.

The method 113 may also include a step 127 (Track Information) directed at tracking deals emerging from the event. Step 127 may include: maintaining an electronic database of information as collected during the event and maintaining written, electronic, or telephonic communications with at least 1 event participant.

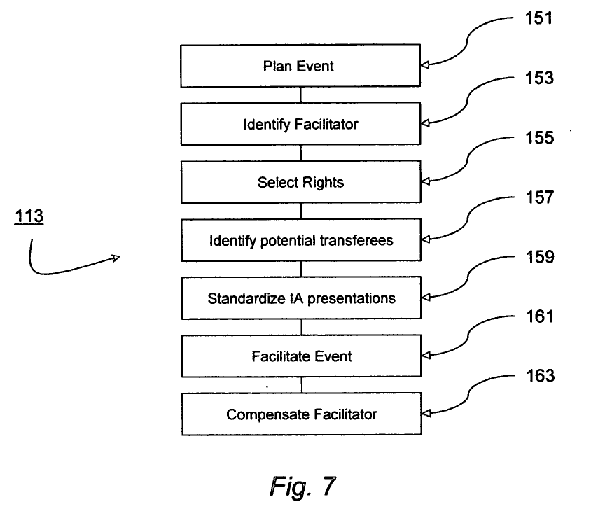

FIG. 7 illustrates another embodiment of the method 113 of the present invention. In this embodiment, the IA Owner plans an event in step 151, and then identifies a Facilitator in step 153. The IA Owner and Facilitator select the IA rights to be transferred in the event in step 155, identify potential transferees in step 157, standardize the IA presentations in step 159, facilitate the event in step 161 and compensate the Facilitator in step 163.

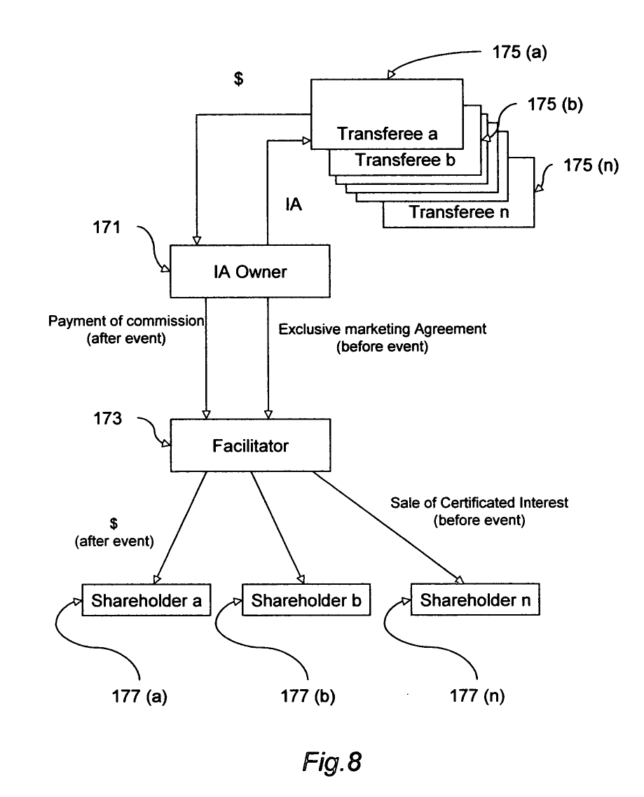

Another embodiment of the present method is illustrated in FIG. 8. Under this business model, the IA Owner 171 engages a Facilitator 173 to help locate Potential Transferees 175 (a)-(n) for the rights to IA offered at an event to be conducted by the Facilitator 173. The Facilitator 173, also called the event managing party, may be provided with an incentive to charge a fee associated with the event based on a percentage of the event proceeds. In this manner, the owner of the IA Owner 171 incurs minimal costs up front, and the ultimate commission paid to the Facilitator 173 is contingent and based on a percentage of the event proceeds. To provide cash in advance of the event, Facilitator 173 may sell interests in the potential event proceeds, which may be certificated to facilitate the creation of a market for these assets, to one or more shareholders 177 (a)-(n) who may then resell their certificated interests to others prior to the event.

To earn the fee, the Facilitator 173 typically helps the IA Owner 171 to identify and package assets to be sold at the event and optimizes the assets by packaging together assets for particular purposes. The Facilitator 173 also identifies potential purchasers by matching up potential purchasers having an interest in assets of the type offered by the IA Owner 171. The Facilitator 173 may also standardize the presentation of information pertaining to the assets offered for sale to facilitate their evaluation by potential purchasers. Facilitator 173 may also value the assets offered for sale in any appropriate manner and provide this information to the owner and/or potential purchasers. The Facilitator 173 may also schedule, publicize, and conduct the event itself, such as an auction. Facilitator 173 may also handle bidding, conflict resolution, escrow, and closing of legal title in connection with the sale. The Facilitator 173 may also track deal flow and maintain a database of information pertaining to completed, planned, and potential events.

What has been described and illustrated herein is a preferred embodiment of the invention along with some of its variations. The terms, descriptions and figures used herein are set forth by way of illustration only and are not meant as limitations. For example, the steps illustrated in FIGS. 1-8 may be performed in an order different from the order shown and/or may be performed simultaneously. Those skilled in the art will recognize that these and many other variations are possible within the spirit and scope of the invention, which is intended to be defined by the following claims—and their equivalents—in which all terms are meant in their broadest reasonable sense unless otherwise indicated.

1. A method of marketing the transfer of specific rights to at least one intellectual asset of a transferor comprising the steps of:
(a) selecting the rights to be transferred by the transferor;
(b) identifying at least one potential transferee of said rights;
(c) preparing a presentation targeted to said potential transferee;
(d) conducting an event wherein said transferor is introduced to the transferee and said presentation is presented to the transferee.

2. The method of claim 1 wherein the step of selecting the rights to be transferred comprises:
(a) analyzing all of the intellectual assets of the transferor;
(b) identifying the business objective of the transferor;
(c) selecting the intellectual assets to be marketed; and
(d) identifying the rights to be transferred consistent with the business objectives of the transferor.

3. The method of claim 1 wherein the step of identifying a transferee comprises:
(a) analyzing
(b) developing criteria
(c) selecting potential transferees that meet criteria

4. The method of claim 1 wherein the step of preparing a presentation to a transferee comprises:
(a) developing a visual layout of information about the intellectual assets
(b) harmonizing the scope of disclosure of the intellectual assets to a consistent standard
(c) harmonizing the delivery method of the presentation to a consistent format

5. The method of claim 1 wherein the step of conducting an event comprises:
(a) planning an event;
(b) scheduling the event at a predetermined time
(c) coordinating the participation of the transferor and the potential transferee at the predetermined time.

6. The method of claim 1 wherein the step of conducting an event is performed by a Facilitator.

7. The method of claim 6 further comprising the step of compensating the Facilitator.

8. A method of transferring rights to intellectual assets comprising the steps of:
(a) planning an event where detailed information about the intellectual assets will be presented to potential transferees;
(b) identifying a Facilitator for the event;
(c) selecting the rights to the intellectual assets that are to be transferred in accordance with predetermined criteria;
(d) identifying potential transferees;
(e) developing standardized presentations having information about the intellectual assets; and
(f) holding an event where the presentations are presented to the potential transferees.

9. The method of claim 8 further comprising the step of compensating the Facilitator.

10. The method of claim 9 further comprising the step of selling shares in the Facilitator's compensation to third parties.

11. The method of claim 9 wherein said step of compensating the Facilitator comprises one or more of the following:
(a) requiring the owner of the intellectual asset to pay an event management fee;
(b) requiring a participant in the event to pay an attendance fee;
(c) requiring a sponsor of the event to pay a sponsorship fee;
(d) requiring the intellectual asset owner to pay a percentage of the proceeds of a transaction as a fee.
